(19) United States
(12) Patent Application Publication
(10) Pub. No.: US 2022/0165391 A1
(43) Pub. Date: May 26, 2022

(54) MULTI-STAGE TREATMENT RECOMMENDATIONS

(71) Applicant: Kyndryl, Inc., New York, NY (US)

(72) Inventors: Caroline K. CAMERON, Columbia, MO (US); Benjamin WISNEWSKI, Columbia, MO (US)

(21) Appl. No.: 17/104,045

(22) Filed: Nov. 25, 2020

(51) Int. Cl. G16H 20/70; G16H 50/20

(52) U.S. Cl. CPC G16H 20/70; G16H 50/20

(57) ABSTRACT

The present disclosure provides for multi-stage treatment recommendations via analyzing a plurality of regimens for treatment of a condition based on a plurality of historical assessments of a plurality of patients who were treated using at least one of the plurality of regimens, wherein sets of historical assessments of the plurality of historical assessments track individual patients of the plurality of patients over time through changes in selected regimens of the plurality of regimens for the individual patients resulting in changes in levels of dysfunction; receiving a new assessment associated with a given patient; scoring, via a machine learning tool, the new assessment based on values for a plurality of variables included in the new assessment; and recommending a given regimen of the plurality of regimens for the given patient based on a category of the plurality of regimens that the plurality of ranked variables place the given patient into.

### BACKGROUND

The present disclosure relates to medical diagnoses, and more specifically, to examples related to the diagnosis and recommendation of treatments for Post-Traumatic Stress Disorder (PTSD) and other mental disorders subject to multi-stage or ongoing treatment. PTSD is a mental health condition triggered by traumatizing events, which may be accompanied by physical trauma, such as a Traumatic Brain Injury (TBI), although a physical trauma is not present in all cases. Symptoms of PTSD can present in response to a triggering event, such as an event that reminds the person living with PTSD of the initial trauma inducing event, and can cause flashbacks, nightmares, severe anxiety, etc., which can interfere with the person's daily life. Treatment of PTSD is a long-term process that can include myriad therapies, lifestyle changes, and pharmaceuticals that are adjusted as the person's condition improves, worsens, or merely progresses without noticeable change over time.

### SUMMARY

According to one embodiment of the present disclosure, a method is provided that includes analyzing a plurality of regimens for treatment of a neurological condition based on a plurality of historical assessments of a plurality of patients who were treated using at least one of the plurality of regimens, wherein sets of historical assessments of the plurality of historical assessments track individual patients of the plurality of patients over time through changes in selected regimens of the plurality of regimens for the individual patients resulting in changes in levels of dysfunction of Activities of Daily Living (ADL); receiving a new assessment associated with a given patient; scoring, via a machine learning tool, the new assessment based on values for a plurality of variables included in the new assessment; and recommending a given regimen of the plurality of regimens for the given patient based on a category of the plurality of regimens that the plurality of ranked variables place the given patient into.

According to one embodiment of the present disclosure, a computer-readable storage medium is provided that includes instructions that when executed by a processor, enable performance of analyzing a plurality of regimens for treatment of a neurological condition based on a plurality of historical assessments of a plurality of patients who were treated using at least one of the plurality of regimens, wherein sets of historical assessments of the plurality of historical assessments track individual patients of the plurality of patients over time through changes in selected regimens of the plurality of regimens for the individual patients resulting in changes in levels of dysfunction of Activities of Daily Living (ADL); receiving a new assessment associated with a given patient; scoring, via a machine learning tool, the new assessment based on values for a plurality of variables included in the new assessment; and recommending a given regimen of the plurality of regimens for the given patient based on a category of the plurality of regimens that the plurality of ranked variables place the given patient into.

According to one embodiment of the present disclosure, a system, is provided that includes a processor; and a memory including instructions that when executed by the processor enable the processor to analyze a plurality of regimens for treatment of a neurological condition based on a plurality of historical assessments of a plurality of patients who were treated using at least one of the plurality of regimens, wherein sets of historical assessments of the plurality of historical assessments track individual patients of the plurality of patients over time through changes in selected regimens of the plurality of regimens for the individual patients resulting in changes in levels of dysfunction of Activities of Daily Living (ADL); receive a new assessment associated with a given patient; score, via a machine learning tool, the new assessment based on values for a plurality of variables included in the new assessment; and recommend a given regimen of the plurality of regimens for the given patient based on a category of the plurality of regimens that the plurality of ranked variables place the given patient into.

### BRIEF DESCRIPTION OF THE SEVERAL VIEWS OF THE DRAWINGS

FIG. 1 illustrates a computing environment in which a multi-stage diagnosis tool is provided in a first cloud network, according to embodiments of the present disclosure.

FIG. 2 is a flowchart of a method for providing multi-stage treatment recommendations, according to embodiments of the present disclosure.

FIG. 3 illustrates a computing system, according to embodiments of the present disclosure.

FIG. 4 depicts a cloud computing environment, according to embodiments of the present disclosure.

FIG. 5 depicts abstraction model layers, according to embodiments of the present disclosure.

### DETAILED DESCRIPTION

The present disclosure provides a multi-stage diagnosis tool for medical professionals to evaluate and treat persons with PTSD and other neurological disorders subject to multi-stage treatment. The multi-stage diagnosis tool collects data related to past trauma history, genetic markers and genes noting predisposition to PTSD, brain scans with structural alterations aligned with PTSD, preliminary assessment results, and medical research data to create a unique patient profile. Causes, or triggers, of symptoms and treatment options are added to the multi-stage diagnosis tool to determine correlations and responses. The multi-stage diagnosis tool generates a score that is classified with a tiered index system of treatments categorized by success rate and relationship to the patient profile. Network monitoring with feedback loops provides further data. This multi-stage diagnosis tool is able to isolate each variable's contribution to treatment efficacy and thereby aid medical professionals to be more effective at diagnosing and treating PTSD with patient profiles of physical and psychological alterations consistent with the disorder and ability. The multi-stage diagnosis tool thereby provides improvements to the treatment process, providing faster and more accurate evidence-based treatments to improve the speed and efficacy of patient results.

Because neurological conditions can be treated by (or affected by) several different medical professionals for one patient (e.g., a psychologist, a therapist, a general practitioner, etc.), and the effects of the selected treatment regimen may not be apparent until a later visit to a medical professional (who may not be associated with the originally prescribing medical professional), the multi-stage diagnosis tool operates across several platforms with semi-anonymized data. The multi-stage diagnosis tool described herein provides enhanced data security and confidentiality when handling a statistically significant dataset across myriad provider networks and provider-selected cloud networks.

The present disclosure provides a solution with examples primarily given for PTSD diagnoses but contemplates that other neurological conditions can benefit from the same or similar analysis. PTSD is often referred to as an invisible wound and it is misunderstood. The present disclosure provides for the evaluation and characterization of the background, trauma, and symptoms of the patient, from a biopsychosocial approach, so that relationships between antagonistic environmental triggers and symptoms can be identified and a differentiation can be made between a highly functioning patient versus a patient with debilitating symptoms. The systems and methods of the present disclosure distinguish each patient diagnosed with PTSD based on symptom presentation, severity, and dysfunction and accumulate empirical data of more evidence-based treatment options and treatment efficacy thereof given the identified characteristics. The systems and methods described in present disclosure further isolate the contribution of the severity and dysfunction level of each symptom and environmental trigger on treatment success to thereby classify PTSD via symptom presentation and trigger interference and to create a tiered index system of evidence-based treatment plan options. The empirical data accumulated assists medical professionals in better classification, treatment, and research of PTSD.

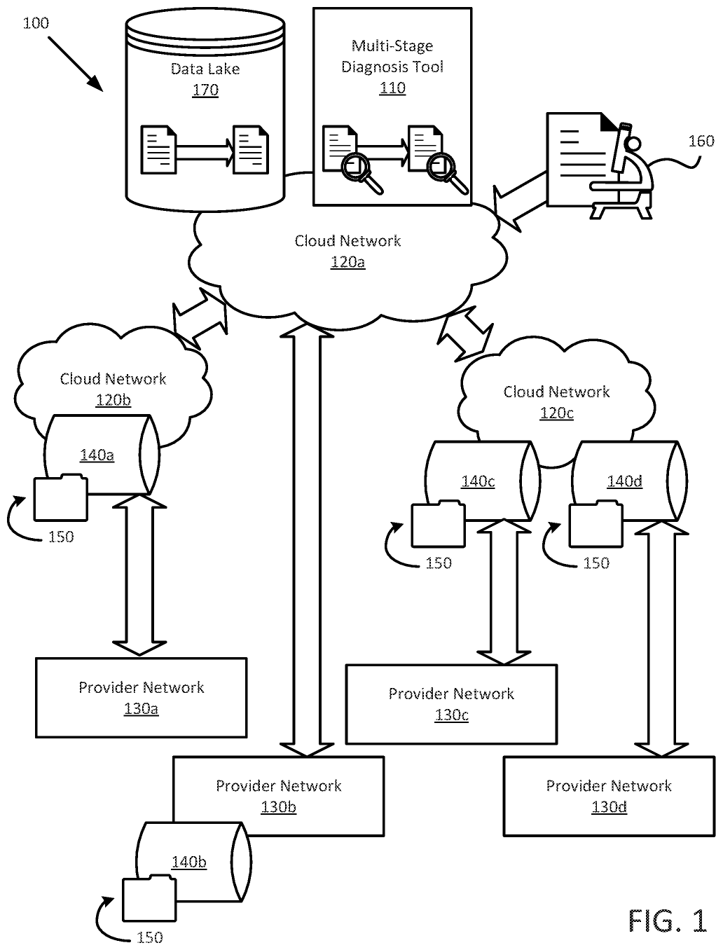

With reference now to FIG. 1, a computing environment 100 in which a multi-stage diagnosis tool 110 is provided in a first cloud network 120a (generally or collectively, cloud network 120), according to embodiments of the present disclosure. The computing environment 100 is shown to include a first provider network 130a (generally or collectively, provider network 130), a second provider network 130b, a third provider network 130c, and a fourth provider network 130d, which are illustrative of local or multi-site networks for individual healthcare providers (e.g., a doctor's office, a practice group, a hospital). Additionally, a second cloud network 120b and a third cloud network 120c are shown, where each cloud network 120a-c is offered by a different cloud network provider.

Various record databases 140a-d (generally or collectively, record databases 140) are shown that include respective pluralities of patient electronic medical records (EMRs) 150 detailing patient data collected during a visit with a medical provider, treatment data for the patient, and the like. Various healthcare providers use different EMR systems, which can result in the EMRs from one provider being formatted differently than the EMRs of another provider. For example, a first EMR system may have more or fewer fields in a record, of the fields may be presented in a different order. Additionally, each EMR system can provide a different identification number for a given patient; complicating cross-provider identification of patients, particularly as healthcare providers move away from using government issued identifiers (e.g., social security numbers, driver license numbers) out of privacy and data security concerns. The individual record databases 140a-d may be hosted locally on a provider networks 130, remotely in a cloud network 120 (including the first cloud network 120a providing the multi-stage diagnosis tool 110), and combinations thereof.

The EMRs 150 can include various fields that are filled in by the providers during a visit with a patient via dropdown menus, radio buttons, or the like, which ensure proper data formatting, but can also include various free-form fields. The free-form fields can accept natural language inputs to describe the patient visit, treatment regimen assigned, and the like to thereby provide the necessary information to describe, in a human-readable format, how the patient is progressing through treatment. Each EMR 150 can also be referred to as a “note” and pertains to one visit at a given medical provider for the given patient. Accordingly, the EMR system correlates several EMRs 150 for a given patient across several visits in the associated record database 140.

The various record databases 140 can include various EMRs 150 related to the patients experiencing the neurological condition who have consented to their data being shared with the multi-stage diagnosis tool 110 as well as patients who have not yet indicated consent. Additionally, the EMRs 150 for various patients who are not experiencing the neurological condition can be included in the record databases 140. Accordingly, the multi-stage diagnosis tool 110 is selective in which EMRs 150 are accessed from the record databases 140 to thereby only handle patient data for patients experiencing the neurological condition who have provided consent according to and applicable with relevant laws and regulations for medical information access and handling in the jurisdictions in which the multi-stage diagnosis tool 110 is deployed.

The multi-stage diagnosis tool 110 extracts information from the EMRs related to the patient, the treatment regimen selected for the patient, and the assessments of how the patient is responding to treatment regimens. In various embodiments, the multi-stage diagnosis tool 110 extracts formatted data as well as free-form natural language data related to the patients and the treatment regimens via a machine learning natural language text extraction tool. Accordingly, notes provided by the medical professionals related to the patients' treatments are extracted for analysis.

The multi-stage diagnosis tool 110 uses semi-anonymized data so that individual patients' data are not revealed, but so that aggregate data and patterns in treatment and outcomes are collected for analysis. For example, the multi-stage diagnosis tool 110 does not extract Personally Identifiable Information (PII), such as name, addresses, governmental identification numbers, or the like, from the EMRs 150. Instead, the multi-stage diagnosis tool 110 links an anonymous key value with each patient so that a set of assessments, collected over time from one or more medical professionals, are gathered and analyzed for a given patient to track changes in treatment regimen and any resulting changes in levels of dysfunction related to activities of daily living (ADL). For example, a set of assessments for a first patient can be gathered over time from a first provider (e.g., a psychologist) and a second provider (e.g., a general practitioner) to aid a third provider in treating a second patient suffering a similar set of symptoms and having a similar patient profile.

When diagnosing a patient for a neurological condition, a physician or other medical professional may compare the symptoms exhibited by the patient against a checklist, such as the Diagnostic and Statistical Manual of Mental Disorders (generally, the DSM). Such checklists for PTSD may include verification of at least one stressor (i.e., exposure or witnessing a traumatic event), at least one intrusion symptom (e.g., upsetting memories, nightmares, flashbacks, emotional distress or physical reactions in response to a triggering event), at least one avoidance behavior related to the trauma, at least two negative alterations in cognition/mood (e.g., inability to recall key details of the trauma, overly negative thoughts or assumptions about self or world, exaggerated blame assessment for the trauma, negative affect or difficulty experiencing positive effect, anhedonia, feelings of isolation), evidence of alterations in reactivity (e.g., irritability or aggression, risky/self-destructive behaviors, hypervigilance, heightened startle reaction, difficulty concentrating, difficulty sleeping), and that the symptoms have lasted for at least one month causing significant distress or functional impairment that cannot be attributed to medication, substance use, or another illness. As will be appreciated, the one-month duration and non-attribution criteria specified by the DSM can lead to delays in treatment and under-reporting (and thus non-treatment) of PTSD, and the strict presentation requirements for the symptoms lack nuance in detection, diagnosis, and treatment of the underlying condition. Additionally, the diagnosis criteria specified by the DSM, and other checklist-based diagnosis tools, merely looks at a single instant in time; as symptoms come and go, worsen or improve, a physician may misdiagnose the neurological condition and fail to identify nuances in the condition. Furthermore, check-list based diagnoses for the presence or absence of a neurological condition provide a binary determination, whereas patients may experience the neurological condition on a spectrum that requires different approaches to treatment at different stages of the condition for different portions of the spectrum.

In contrast to a check-list based diagnosis system, the multi-stage diagnosis tool 110 extracts several values for different variables from the EMRs 150 to build a patient profile to identify various groups of patients into categories on a spectrum. For example, the multi-stage diagnosis tool 110 can evaluate an ADL level as dysfunctional, moderately impacted, minimally impacted, or having no impact, and assign a weighted score to each ADL level. Similarly, the multi-stage diagnosis tool 110 can identify various variables and determine various spectra for current symptoms, medical history, past trauma history, a DNA or genetic profile, a brain scan (e.g., Magnetic Resonant Imaging (MRI), x-ray, etc.), assessment survey, resiliency training, previously adopted coping strategies, etc., and assign various weighted scores to those categories based on analysis of the extracted data. Because the notes include natural language text, and might not recite a specific category (e.g., “dysfunctional ADL level”) or may recite a specific category in the negative when performing an analysis or indicating an improvement (e.g., “no nightmares” or “nightmare frequency diminishing”), multi-stage diagnosis tool 110 performs a heuristic analysis to determine what value to assign to each variable.

Using the values assigned to each variable, the multi-stage diagnosis tool 110 matches potential treatments to address the symptoms and causes of the neurological condition. In various embodiments, an algorithmic score for the patient is matched to a tiered score index system for one or more recommendation to the medical professional for the patient to follow in parallel or in series. The symptoms can include those identified as related to the neurological condition (e.g., via the DSM), as well as symptoms from other conditions also experience by the patient both related to the trauma (e.g., a TBI from the source of trauma) and not directly related, which may aggravate the neurological condition or affect available treatment options. In various embodiments, these individual treatments are grouped into regimens that include various individual treatments such as, for example, therapy, medication, creative expression programs (e.g., art therapy), clinical trials, service animals, lifestyle changes, physical therapy or exercise programs, and the like.

In some embodiments, the EMRs 150 include visual data and lab report data. For example, the presence of PTSD can be physically documented by structural alterations in the brain visible on an MRI (Magnetic Resonant Imaging) scan. This is an important addition to an overall evaluation since PTSD has carried the alias of “the invisible wound” and has been subject to scrutiny. A brain scan, such as an MRI, provides physical documentation of the structural alterations that are indicative of the presence of PTSD.

In addition to the EMRs 150, the multi-stage diagnosis tool 110 can examine research papers 160 for experimental treatments undergoing clinical trials. The research papers 160 identify cohorts of patients undergoing experimental treatments with various levels of efficacy in the results. As will be appreciated, researchers may divide groups of patients undergoing the trial into various cohorts based of various features (e.g., persons of a given demographic, having a given trauma history, having tried a previous treatment, etc.), and a control group that does not receive the experimental treatment (or a placebo) may also be included against which an efficacy level is judged. When an efficacy level of an experimental treatment rises above a given threshold, or when a stall (or regression) in a given patient's current treatment regimen is noted, the multi-stage diagnosis tool 110 can recommend the experimental treatments be included in or substitute for the patient's next treatment regimen. In various embodiments, before recommending the inclusion of an experimental treatment from a clinical trial, the multi-stage diagnosis tool 110 verifies whether the given patient is in a cohort that underwent the clinical trial with a threshold level of efficacy.

In various embodiments, the multi-stage diagnosis tool 110 collects the patient data and research data to develop a data lake 170 stored in the first cloud network 120a that hosts the multi-stage diagnosis tool 110. The data lake 170 provides a statistically significant pool of patients and treatment histories for forming the recommendations provided to the various medical providers. The data stored in the data lake 170 are semi-anonymized so that records for one patient are identifiable as being related to that single patient but cannot be traced back to PII for that patient without a key. In various embodiments, the medical practitioners submit data or request diagnosis recommendations along with the key so that newly submitted assessments are grouped with historical assessments for the same patient and recommendations are based on the ongoing treatment record for that patient.

When a new assessment for a patient is received (e.g., during a visit to a medical professional), the multi-stage diagnosis tool 110 draws from the data lake 170 and the newly received assessment to provide an updated recommendation for the treatment regimen to provide to the patient. The multi-stage diagnosis tool 110 also identifies which historic assessments are related to the new assessment (e.g., are for the same patient), and updates the historic set based on new data. For example, the multi-stage diagnosis tool 110 notes whether symptoms have improved, worsened, or stayed the same between visits, and updates the values assigned to the various variables tracked for the patient. The multi-stage diagnosis tool 110 then reevaluates and re-ranks symptoms and the causes of those symptoms to determine correlations of causes to the regimen's efficacy. The correlations are then used for different patients so that patients can be helped via the experiences of other patients who belong to a same category as an effectively treated patient.

In various embodiments, in addition to providing data for the multi-stage diagnosis tool 110, some or all of the data lake 170 is provided to, or made accessible to, various research entities for further analysis. For example, a Department of Defense, Veterans Affairs, or Firefighters Union researcher can access the data lake 170 to identifying clusters of persons and associated traumas to identify at-risk groups, likely-inducing traumas, or the like. These data from the data lake 170 can be combined with other databases by the researcher to further identify persons (and attributes thereof) who experience similar traumas with no or more-minor diagnoses for the neurological conditions to thereby reduce the occurrence or severity of the condition in the future. For example, a Veterans Affairs researcher may be able to identify from the data lake 170 that patients with longer (but fewer) or shorter (but longer) deployments exhibit different ADL levels to recommend changes in how deployments are structured. In another example, a Firefighters Union researcher can identify that persons given unrestricted time-off from work after experiencing or witnessing a traumatic event are more likely to develop higher ADL levels than persons who are given structured time-off or low-stress work afterwards and thereby change how (and when) support programs are provided to firefighters.

Categorizing the patients based on the incoming data allows for a first patient, who experienced a trauma of type X, has symptoms with severity Y, and demographic details Z to undergo successful treatment for the neurological condition, and for that success to be used by a second patient who also experience trauma of type X, symptoms of severity Y, and demographic details Z to be assigned the same (or a similar) treatment regimen. Because treatment for neurological conditions can take a long time to show positive effects, knowing that a similarly effected person received positive results can aid in knowing when a treatment is stalled or ineffective, or is merely in an expected holding period before an improvement. For example, by knowing that a similarly effected patient went to therapy for several months before showing signs of improvement, a medical practitioner can recommend that another patient should stick with a therapy program—or switch to a different therapist—before switching to a different regimen that does not include therapy.

One benefit of the multi-stage diagnosis tool 110 is that by revealing the distinction and broad range in PTSD symptom presentation, both patients and medical professionals can better understand and treat PTSD. This is particularly important as two patients with the same blanket PTSD diagnosis can be affect in vastly different ways. For example, a first patient can maintain a highly functioning life while a second patient is debilitated. By refining a PTSD into a spectrum diagnosis, persons who are affected differently, with different sources of PTSD, have their PTSD triggered differently, etc. can be treated with different and better-tailored treatment regimens. Additionally, the tiered index or spectrum system of diagnosis can identify treatment options that are particularly effective (or ineffective) for different classes of patients so that a broader range of treatments can be targeted to the most receptive patients.

The multi-stage diagnosis tool 110 uses network monitoring with feedback loops to accumulate the empirical data on treatment efficacy in the databases to provide more ongoing evidence-based treatment options to medical providers.

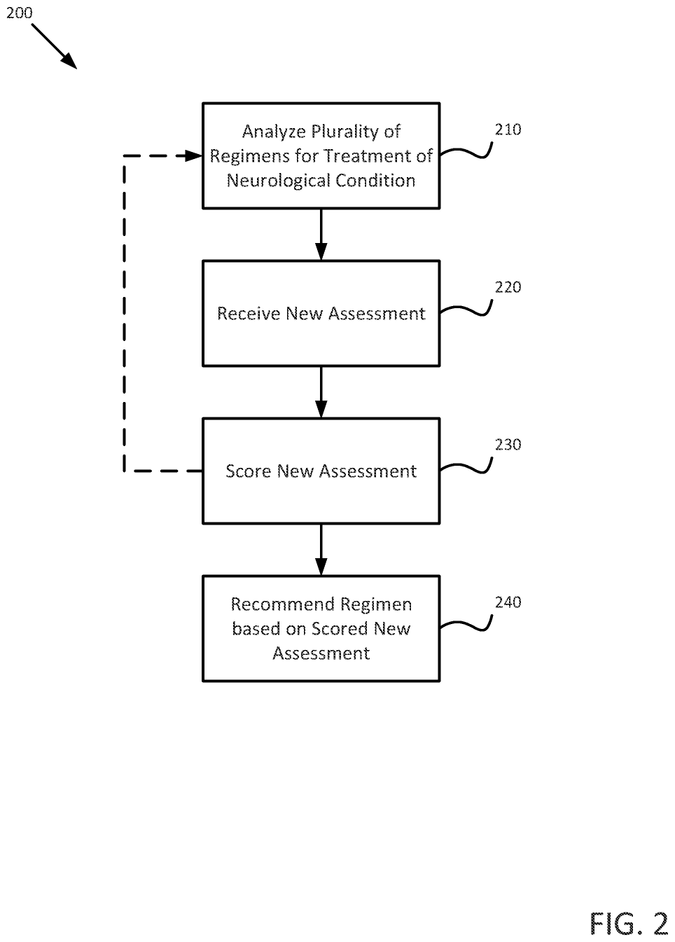

FIG. 2 is a flowchart of a method 200 for providing multi-stage treatment recommendations, according to embodiments of the present disclosure. Method 200 begins at block 210, where the multi-stage diagnosis tool analyzes a plurality of regimens for treatment of a neurological condition, such as PTSD. The analysis is based on a plurality of historical assessments of a plurality of patients treated for the neurological condition, in which sets of historical assessments of individual patients track the treatment of the neurological condition over time. These sets of historical assessments provide for a history that shows changes in the regimens selected for treating the individual patients and the level of dysfunction that the neurological condition imposes on those individual patients. Accordingly, the multi-stage diagnosis tool builds and analyzes a data set that identifies and categorizes various patients in association with the treatments prescribed to those patients to monitor and correlate changes in the treatment regimens to changes in the level of dysfunction due to the neurological condition.

At block 220, the multi-stage diagnosis tool receives a new assessment from a medical practitioner that requests a diagnosis recommendation to provide to the associated patient. In various embodiments, the new assessment is processed and added to the data lake; extracting relevant data points, ignoring and discarding irrelevant data points, and semi-anonymizing the record. When the given patient is not initially included in the plurality of patients from whom the plurality of historical assessments was made, the multi-stage diagnosis tool creates a new set of records for that patient, beginning with the initial assessment. When the given patient is already included in the plurality of patients, the multi-stage diagnosis tool uses the new assessment to update a historical assessment associated with that patient to reevaluate an efficacy of an associated regimen for the patient. Stated differently, the multi-stage diagnosis tool builds a historical record for each patient across several assessments taken at different times, and updates the prognosis of the individual patients, which can in turn be used to better diagnose that patient and other patients whose assessments are analyzed.

In various embodiments, a machine learning based natural language text analysis tool extracts data from electronic medical records (submitted by medical professionals treating a patient for the neurological condition and other conditions) and from medical journals or clinical reviews for evaluating treatments for cohorts of anonymous patients. The extracted data can be retrieved from various databases, including local databases hosted at the facilities of medical providers or research organizations, cloud hosted record keeping systems used by medical professionals or research organizations, or cloud hosted databases in the same cloud network as provides the multi-stage diagnosis tool.

At block 230, the multi-stage diagnosis tool scores, via a machine learning tool using a machine learning model, the new assessment based on values for the plurality variables included in the new assessment. In various embodiments, the scoring is performed via a natural language text analysis of the notes submitted by the patient or the medical professional in the new assessment to categorize one or more symptoms. These variables for an associated patient include one or more of a genetic profile (e.g., a DNA or gene analysis, an ethnicity, gender, age, etc.), a psychological profile (e.g., co-presenting depression, generalized anxiety, previously adopted coping strategies, resiliency training, etc.), a trauma profile (e.g., type of event, whether directly or indirectly observed, chronic/single event), a medical profile (e.g., heart conditions, brain scan, current medications), a symptom profile (e.g., level of dysfunction, triggers, duration of attacks, frequency of attacks, etc.), and the like.

At block 240, the multi-stage diagnosis tool recommends a given regimen of the plurality of regimens to the medical provider for the patient for whom the associated request was received. In various embodiments, the multi-stage diagnosis tool recommends one or more treatments to form a treatment regimen for the patient. Based on the reviewed variables from the assessments for the patient, the multi-stage diagnosis tool places the patient into a category with similar patients (i.e., patients having assessments whose variables are equal to or within a weighted threshold of the assessed patient). The effective treatments observed in the category are then identified and provided as recommendations to the medical professional to assign for the given patient's treatment regimen.

In various embodiments, the treatment regimens include various treatments including creative expression programs (e.g., art therapy), service animals, exercise or physical therapy routines, medications, therapy sessions (e.g., with support groups or licensed therapists). The multi-stage diagnosis tool ranks which treatments options to present to the medical professional and what order to recommend the various treatments.

The efficacy of the treatment options, and a measure of how impactful on the patient's lifestyle, are used to determine which treatment options to recommend for or recommend against. For example, having a trained service dog may be supported in the evidence (via research and collected patient data) to be very effective for a first patient, but may have a high-impact on the first patient's lifestyle (e.g., caring for a dog, an allergy to dogs, etc.), and may therefore be rated as a lower-priority treatment than for a second patient who already owns a non-service dog. In another example, using the same dataset indicating the general efficacy of support animals, the multi-stage diagnosis tool identifies that assigning a service animal to a third patient would be inappropriate if the trauma causing in the third patient's neurological condition were triggered by animals.

Additionally, as neurological conditions, such as PTSD, require long term treatment and symptom maintenance, the multi-stage diagnosis tool can recommend an order of treatments in the regimen. For example, the data may indicate that a service animal and group therapy are effective treatments but are more effective after individual therapy and medication lower the level of dysfunction, than on their own. Accordingly, the multi-stage diagnosis tool recommends medication and individual therapy until an improvement is seen in the patient, at which point medication and group therapy may be substituted for a service animal and group therapy.

Moreover, as the patient may experience symptoms that vary in intensity over time, the multi-stage diagnosis tool helps provide long term regimen management that ignores temporary swings (e.g., not prematurely stopping, switching, starting, or adjusting a treatment), but identifies stalls or regressions that do warrant adjusting a regimen to address. By observing the standard lengths of time of similar patients to begin exhibiting improvement and the level of variant of symptom presentation over that time period, the multi-stage diagnosis tool can reassure patients and medical professionals that a selected regimen is appropriate and remains effective, despite short term regressions or stalls. Similarly, the multi-stage diagnosis tool identifies when a regression or stall is serious enough to warrant a change. In various embodiments, when a stall or regression satisfies a threshold (e.g., symptoms remain unchanged for at least X months, symptoms worsen to a given level of ADL), the multi-stage diagnosis tool recommends experimental regimens from clinical trials that have a positive cohort that matches the patient's demographics for the experimental regimen at a higher rate to ensure that new effective treatments are provided to the medical professional to consider for the patient.

After providing the medical professional with the recommendation, method 200 may conclude. As will be appreciated, several instances of method 200 may be performed contemporaneously, and each instance may produce different results at block 240 based on the new assessment for the associated patient received at block 220. Additionally, each new assessment analyzed at block 230 is added to the plurality of assessments analyzed at block 210 for later-performed instances of method 200.

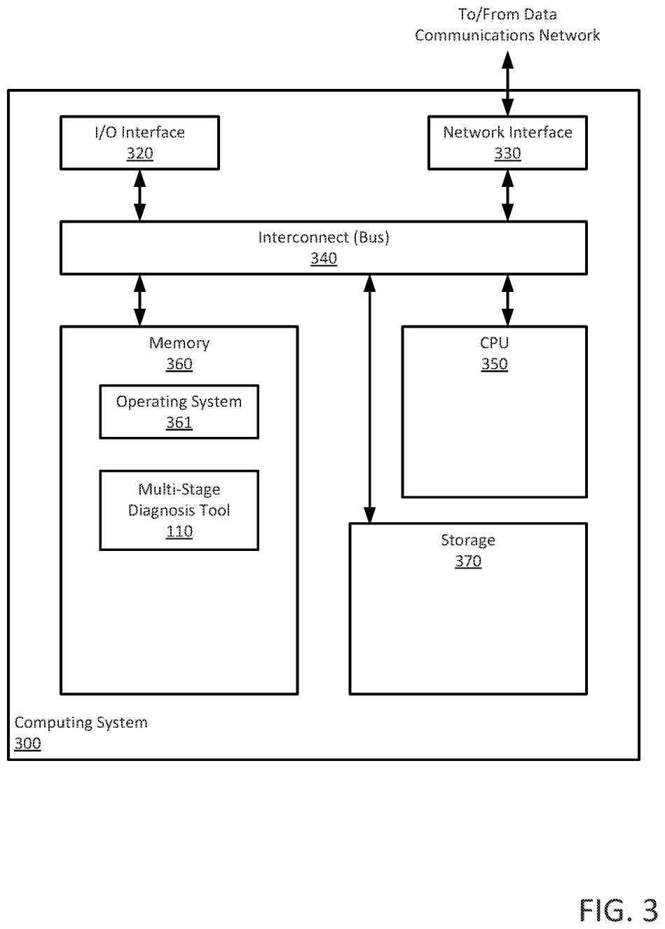

FIG. 3 illustrates a computing system 300, according to embodiments of the present disclosure. As shown, the computing system 300 includes, without limitation, a central processing unit (CPU) 350, a network interface 330, an interconnect 340, a memory 360, and storage 370. The computing system 300 may also include an I/O device interface 320 connecting I/O devices 310 (e.g., keyboard, keypad, display, touchscreen, biometric scanner, and mouse devices) to the computing system 300.

The CPU 350 retrieves and executes programming instructions stored in the memory 360. Similarly, the CPU 350 stores and retrieves application data residing in the memory 360. These instructions are included in an instruction cache 351 for execution and manipulation as described in the present disclosure. The interconnect 340 facilitates transmission, such as of programming instructions and application data, between the CPU 350, I/O device interface 320, storage 370, network interface or other interconnect 340, and memory 360. CPU 350 is included to be representative of a single CPU, a microprocessor, multiple CPUs, a single CPU having multiple processing cores, and the like. And the memory 360 is generally included to be representative of a random access memory. The storage 370 may be a disk drive storage device. Although shown as a single unit, the storage 370 may be a combination of fixed and/or removable storage devices, such as magnetic disk drives, flash drives, removable memory cards or optical storage, network attached storage (NAS), or a storage area-network (SAN). The storage 370 may include both local storage devices and remote storage devices accessible via the network interface 330 (e.g., cloud storage).

Further, although shown as a single computing system, one of ordinary skill in the art will recognized that the components of the computing system 300 shown in FIG. 3 may be distributed across multiple computing systems connected by a data communications network.

As illustrated, the memory 360 includes an operating system 361, which may include one or more file systems, and a set of processor instructions to perform various actions as described herein. These actions may be informed and formatted according to various applications, such as the multi-stage diagnosis tool 110, running in the memory as instructions executed by the CPU 350.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows: On-demand self-service: a cloud consumer can unilaterally provision computing capabilities, such as server time and network storage, as needed automatically without requiring human interaction with the service's provider. Broad network access: capabilities are available over a network and accessed through standard mechanisms that promote use by heterogeneous thin or thick client platforms (e.g., mobile phones, laptops, and PDAs). Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter). Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time. Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows: Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings. Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations. Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows: Private cloud: the cloud infrastructure is operated solely for an organization. It may be managed by the organization or a third party and may exist on-premises or off-premises. Community cloud: the cloud infrastructure is shared by several organizations and supports a specific community that has shared concerns (e.g., mission, security requirements, policy, and compliance considerations). It may be managed by the organizations or a third party and may exist on-premises or off-premises. Public cloud: the cloud infrastructure is made available to the general public or a large industry group and is owned by an organization selling cloud services. Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

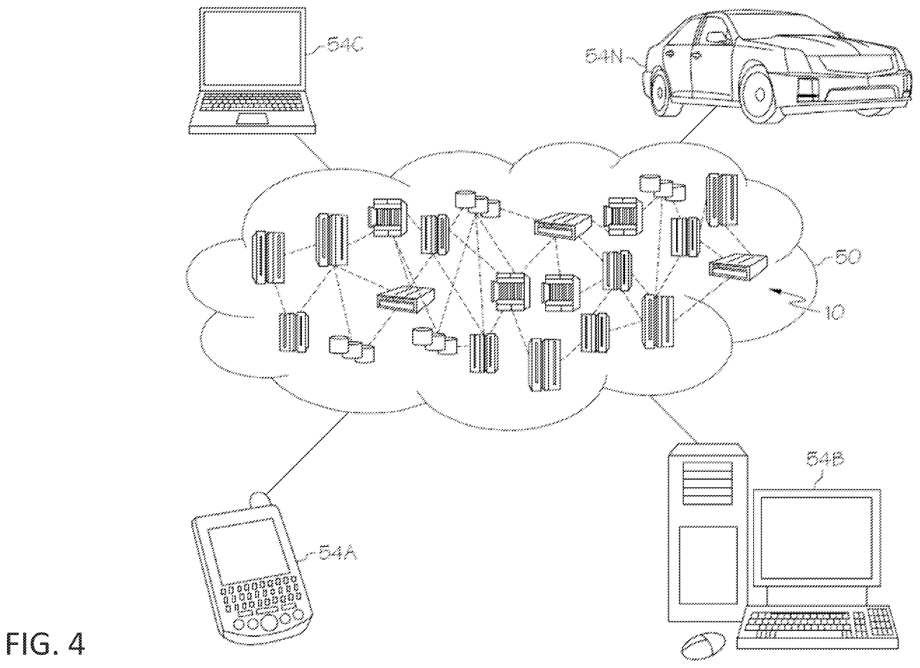

Referring now to FIG. 4, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 10 and cloud computing environment 50 can communicate with any type of computerized device over any type of network and/or network addressable connection (e.g., using a web browser).

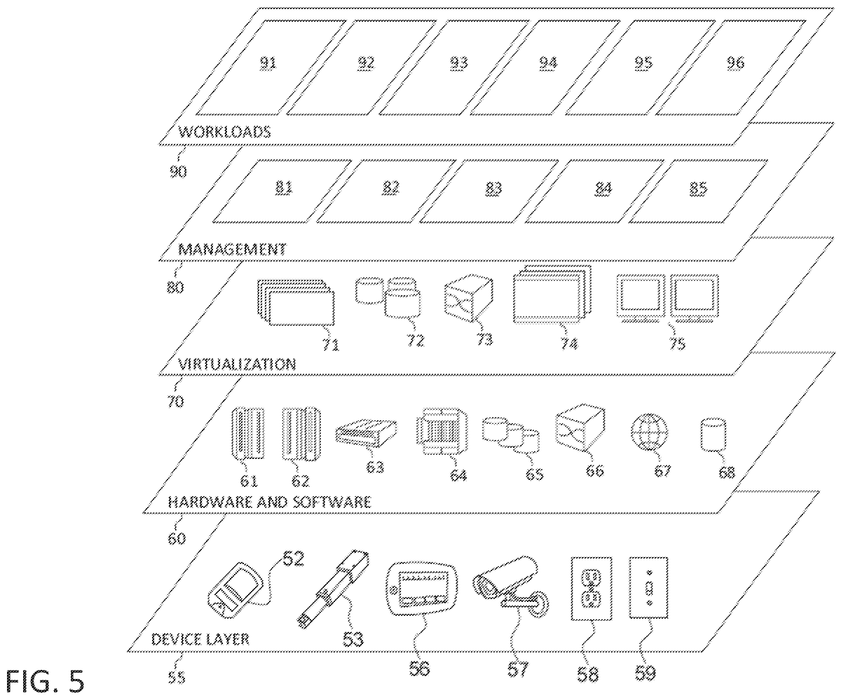

Referring now to FIG. 5, a set of functional abstraction layers provided by cloud computing environment 50 (FIG. 4) is shown. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and class balancing training datasets for intent authoring using search 96.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the preceding, reference is made to embodiments presented in this disclosure. However, the scope of the present disclosure is not limited to specific described embodiments. Instead, any combination of the features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the aspects, features, embodiments and advantages discussed herein are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

Aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.”

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the foregoing is directed to embodiments of the present invention, other and further embodiments of the invention may be devised without departing from the basic scope thereof, and the scope thereof is determined by the claims that follow.

What is claimed is:

1. A method comprising:
analyzing a plurality of regimens for treatment of a neurological condition based on a plurality of historical assessments of a plurality of patients who were treated using at least one of the plurality of regimens, wherein sets of historical assessments of the plurality of historical assessments track individual patients of the plurality of patients over time through changes in selected regimens of the plurality of regimens for the individual patients resulting in changes in levels of dysfunction of Activities of Daily Living (ADL);
receiving a new assessment associated with a given patient;
scoring, via a machine learning tool, the new assessment based on values for a plurality of variables included in the new assessment; and
recommending a given regimen of the plurality of regimens for the given patient based on a category of the plurality of regimens that the plurality of ranked variables place the given patient into.

2. The method of claim 1, wherein the plurality of variables includes:
a genetic profile for an associated patient;
a brain scan for the associated patient;
a psychological profile for the associated patient;
a trauma profile for the associated patient;
a medical profile related to other conditions than neurological condition for the associated patient; and
a symptom profile for the neurological condition for the associated patient.

3. The method of claim 1, wherein the given patient is not initially included in the plurality of patients from whom the plurality of historical assessments was made.

4. The method of claim 1, wherein the given patient is initially included in the plurality of patients, and the new assessment updates a historical assessment of the plurality of historical assessments to reevaluate an efficacy of an associated regimen for the given patient.

5. The method of claim 1, wherein the new assessment and the plurality of historical assessments are extracted, via a machine learning natural language text analysis tool, from electronic medical records submitted by medical professionals treating an associated patient for the neurological condition and other conditions.

6. The method of claim 5, wherein the electronic medical records from which the new assessment and the plurality of historical assessments are extracted are stored in a plurality of databases including:
a locally hosted database for a first medical professional; and
a cloud database hosted for a second medical professional that is hosted in a different cloud network than the machine learning natural language text analysis tool is hosted in.

7. The method of claim 1, wherein the plurality of regimens includes:
creative expression programs;
service animals;
exercise routines;
medications; and
therapy sessions; and
wherein recommending the given regimen of the plurality of regimens for the given patient further comprises ranking plurality of regimens based on efficacy and lifestyle impact.

8. The method of claim 1, wherein analyzing the plurality of regimens for treatment of the neurological condition further comprises:
analyzing an experimental regimen based on medical studies for treatment of the neurological condition; and
wherein the experimental regimen is recommended as the given regimen when the given patient matches a positive cohort categorization for the experimental regimen and a set of historical assessments for the given patient indicate a stall or regression in a level of dysfunction of ADL.

9. The method of claim 1, further comprising:
providing a research entity access to a data lake including the plurality of historical assessments and the plurality of regimens.

10. The method of claim 1, wherein scoring the new assessment recommends a cluster for the given patient based on a diagnosis spectrum for the neurological condition based on a current score and any historic scores for the given patient generated by the machine learning tool.

11. A computer-readable storage medium, including instructions that when executed by a processor, enable performance of:
analyzing a plurality of regimens for treatment of a neurological condition based on a plurality of historical assessments of a plurality of patients who were treated using at least one of the plurality of regimens, wherein sets of historical assessments of the plurality of historical assessments track individual patients of the plurality of patients over time through changes in selected regimens of the plurality of regimens for the individual patients resulting in changes in levels of dysfunction of Activities of Daily Living (ADL);
receiving a new assessment associated with a given patient;
scoring, via a machine learning tool, the new assessment based on values for a plurality of variables included in the new assessment; and
recommending a given regimen of the plurality of regimens for the given patient based on a category of the plurality of regimens that the plurality of ranked variables place the given patient into.

12. The computer-readable storage medium of claim 11, wherein the plurality of variables includes:
a genetic profile for an associated patient;
a brain scan for the associated patient;
a psychological profile for the associated patient;
a trauma profile for the associated patient;
a medical profile related to other conditions than neurological condition for the associated patient; and
a symptom profile for the neurological condition for the associated patient;
wherein the plurality of regimens includes:
creative expression programs;
service animals;
exercise routines;
medications; and
therapy sessions; and
wherein recommending the given regimen of the plurality of regimens for the given patient further comprises ranking plurality of regimens based on efficacy and lifestyle impact.

13. The computer-readable storage medium of claim 11, wherein the given patient is initially included in the plurality of patients, and the new assessment updates a historical assessment of the plurality of historical assessments to reevaluate an efficacy of an associated regimen for the given patient.

14. The computer-readable storage medium of claim 11, wherein the new assessment and the plurality of historical assessments are extracted, via a machine learning natural language text analysis tool, from electronic medical records submitted by medical professionals treating an associated patient for the neurological condition and other conditions.

15. A system, comprising:
a processor; and
a memory including instructions that when executed by the processor enable the processor to:
analyze a plurality of regimens for treatment of a neurological condition based on a plurality of historical assessments of a plurality of patients who were treated using at least one of the plurality of regimens, wherein sets of historical assessments of the plurality of historical assessments track individual patients of the plurality of patients over time through changes in selected regimens of the plurality of regimens for the individual patients resulting in changes in levels of dysfunction of Activities of Daily Living (ADL);
receive a new assessment associated with a given patient;
score, via a machine learning tool, the new assessment based on values for a plurality of variables included in the new assessment; and
recommend a given regimen of the plurality of regimens for the given patient based on a category of the plurality of regimens that the plurality of ranked variables place the given patient into.

16. The system of claim 15, wherein the given patient is not initially included in the plurality of patients from whom the plurality of historical assessments was made.

17. The system of claim 15, wherein the given patient is initially included in the plurality of patients, and the new assessment updates a historical assessment of the plurality of historical assessments to reevaluate an efficacy of an associated regimen for the given patient.

18. The system of claim 15, wherein the new assessment and the plurality of historical assessments are extracted, via a machine learning natural language text analysis tool, from electronic medical records submitted by medical professionals treating an associated patient for the neurological condition and other conditions, wherein the electronic medical records from which the new assessment and the plurality of historical assessments are extracted are stored in a plurality of databases including:
a locally hosted database for a first medical professional; and
a cloud database hosted for a second medical professional that is hosted in a different cloud network than the machine learning natural language text analysis tool is hosted in.

19. The system of claim 15, wherein the plurality of regimens includes:
creative expression programs;
service animals;
exercise routines;
medications; and
therapy sessions; and
wherein recommending the given regimen of the plurality of regimens for the given patient further comprises ranking plurality of regimens based on efficacy and lifestyle impact.

20. The system of claim 15, wherein analyzing the plurality of regimens for treatment of the neurological condition the processor is further enabled to:
analyze an experimental regimen based on medical studies for treatment of the neurological condition; and
wherein the experimental regimen is recommended as the given regimen when the given patient matches a positive cohort categorization for the experimental regimen and a set of historical assessments for the given patient indicate a stall or regression in a level of dysfunction of ADL.
